(19) United States
(12) Patent Application Publication
(10) Pub. No.: US 2010/0004212 A1
(43) Pub. Date: Jan. 7, 2010

(54) DUAL PROTECTION OF MEDICINAL- PHARMACEUTICAL PRODUCTS AND NEW TECHNIQUES FOR THEIR APPLICATION

(76) Inventors: Spyridon Eduardos Tsakas, Athens (GR); Spyridon Tsakas, Athens (GR)

Correspondence Address: Diane Dunn McKay, Esq.;Mathews, Shepherd, McKay & Bruneau, P.A., Suite 201, 29 Thanet Road, Princeton, NJ, 08540, US

(21) Appl. No.: 12/327,947

(22) Filed: Dec. 4, 2008

(30) Foreign Application Priority Data
Jul. 1, 2008 (GR) 20080100446

(51) Int. Cl. A61K 31/60

(52) U.S. Cl. 514/165

(57) ABSTRACT

The present invention solves under the best possible method three problems which preoccupy the production, delivery and use of pharmaceutical, medicinal or other products: A) Their protection from oxidating alterations with the use of reducing or antioxidant substances-mixtures. B) The protection from alterations which are caused by light exposure primarily for light sensitive substances-mixtures and C) Through a special method (hollow caps) the end product gains the protection stated in A and B while simultaneously simplifying the production and safety of the manufactured products in the most economical, efficient, and greatly simplifies their use even in the most remote conditions and areas.

### DESCRIPTION OF THE INVENTION

1. Technical Field of the Invention

The invention refers to and offers a solution to two important problems that concern the production and application of medicine and medicinal products. The first is their protection from exposure to light and oxidations that cause undesirable changes to their features and the second is the improvement of the methods for safer and easier application of them in injectable, drinkable and other forms.

2. Level of the Invention

Products that restore or protect our health come in different forms (capsules, tablets, etc.) and application methods (injectable, drinkable, etc.). They are comprised of active ingredients, excipients, and packaging materials. Their active ingredients are and must be maintained unchanged for as long as possible (proposed usability period of products). The excipients, shape and packaging help in their protection from alterations and create the conditions for the longest possible positive results in the depth of time. All manufactured products pharmaceutical, medicinal, diagnostic, and veterinary undergo modifications either small or large with the resulting limited usability period according to the methods of production and preservation. The level of this process therefore always requires improvement, and that is what this patent promises to offer to a great extent.

3. Object of the Invention

The most crucial factors which alter pharmaceutical, medicinal and other products are four, and sometimes can be correlated: 1) Oxygen, 2) Humidity, 3) Temperature, 4) Light. The object of this invention refers to the protection from oxidations, and light exposure. Therefore this invention is meant for light sensitive and oxidation sensitive products and primarily to the protection of their active ingredients.

The protection of products from the humidity factor, currently occurs through: a) their production in the driest of possible conditions, b) addition in the end product of an envelope containing moisture absorbing substances, c) replacement of the contained air (within the dry phase of the vial) with dry, neutral gasses such as Nitrogen, Carbon dioxide, etc.

The latter mentioned case protects also from possible oxidations by removing the contained within oxygen and replacing it with neutral gasses.

The present patent refers primarily to the protection from oxidizing alterations with the use of antioxidant substances either within the dry phase of the containers (i.e. capsules in the form of excipients), liquid phase, or in an envelope within the final product so that the oxygen/oxy free radicals present or created within are bonded by the antioxidant substances, thus oxidizing the molecules of the reducing substance and making the active substance and its basic excipients remain unaffected by oxidizing alterations.

The antioxidant protection depends on the oxidative dynamic difference between our antioxidant factor and the active ingredients-excipients and their corresponding comparative amounts present (oxygen, antioxidant active ingredient, excipients, etc).

We state here a multitude of reducing (antioxidant) factors which we also declare through this patent for specific uses. As well as other natural or synthetic factors which will be isolated, manufactured, and be used as described here. In this way, based on the ingredients of each product we can select a factor or combination of factors from: ascorbic acid and its salts (vitamin C), glutathione (GSH), Vitamin E, Uric acid, Urea, L-phenylalanine, L-histidine, Mannitol, Salicylic acid, Acetylsalicylic acid (aspirin), DMSO (dimethylsulfoxide), Tocopherols. Reducing enzymes such as SOD (superoxidase dismutase), Glutatnione reductase, Catalase, Amino acids or reducing micropeptides, Cysteine, Cystine, Glutamine, Glutaminic, Cytochromes, Selinium (Se) and its bonds, Chelate bonds such as EDTA which bond iron ions Fe++ (Fenton reaction) and copper Cu++.

### DETAILED DESCRIPTION OF THE INVENTION

The invention describes a pharmaceutical and/or medicinal product which is destined for humans or animals, and is applied in an injectable, drinkable, liquid or dry form which product contains at least one active substance, as well as an antioxidant factor that is selected because it functions to protect it from oxidation.

Furthermore, if the pharmaceutical/medicinal product described by the invention has both a dry and liquid phase, then the antioxidant factor can be in the dry phase, the liquid phase, or both.

The invention also describes the use of an antioxidant factor in a pharmaceutical/medicinal product for the protection of each active ingredient, its excipients and/or its packaging from oxidations, where the choice of the antioxidant factor depends on the type, quantity, and packaging of the active ingredient or its excipients, that require the antioxidant protection we desire.

One application of this invention, involves the addition of an antioxidant factor within the final product ready for use, or within the final package in a small envelope.

Additionally, the envelope containing the antioxidant factor described in the invention is air permeable in order to bond the oxygen/oxy free radicals contained within the package of the product necessitating protection.

According to the application of this invention, the container of the antioxidant factor can be in the form of either an envelope or capsule.

The antioxidant factor which this invention describes can be selected from:
ascorbic acid and its salts (vitamin C)
glutathione (GSH)
Vitamin E
Uric acid
Urea
L-phenylalanine
L-histidine
Salicylic acid
Acetylsalicylic acid (aspirin)
DMSO (dimethylsulfoxide)
Mannitol
Tocopherols
Reducing enzymes such as SOD (superoxidase dismutase)
Glutatnione reductase
Amino acids or reducing micropeptides
Cysteine
Cystine
Glutamine
Glutaminic
Cytochromes
Selinium (Se) and its bonds
Chelate bonds such as EDTA which bond iron ions Fe++ (Fenton reaction) and copper Cu++
   or a combination of the above.

In another application of this invention, the container has a hollow non-light transparent cap for the protection of the active ingredient, its excipients, and the antioxidant factor from oxidations, light exposure and UV radiation while also having a dark colored vial to protect the solution.

Moreover, the container can be used for the injectable application of products sensitive to alterations, such as i.e. LAS (lysine acetylsalicylate) and requires the use of only one container, contrary to the two currently needed for it.

In an additional use of this invention, the vial is used for the application of medicines, such as antibiotics, vaccines, antidotes which once again requires the use of only one vial that contains both the dry and liquid phases together, making its use ideal for remote areas/countries, sea excursions, explorations, hikes, and military expeditions.

### Application Example

The molecular changes of salicylic acid as well as its derivative acetylsalicylic acid (aspirin) caused by hydroxy radicals has as a result the creation of benzoic hydroxylated compounds. These insoluble hydroxy derivatives of salicylic acid and aspirin could take place also in pharmaceutical products. Similar decompositions due to oxidating factors may occur when the soluble and pain alleviating lysine acetyl salicylate salt (LAS) is present in a product. LAS (included in Merck Index 2006, page 964 under number 5668) is used as an injectable pain alleviator due to its high solubility and effectiveness. This salt when oxidized and/or exposed to light may break down in the dry phase of the product into its two main components and other oxidized by products; the insoluble acetylsalicylic acid, lysine, and the by products so when the mixture of the dry phase with the liquid phase are mixed the insoluble acetylsalicylic acid precipitates. Due to the aforementioned alterations the final mixture is unsuitable and possibly hazardous for injection.

Therefore the antioxidant protection of products containing LAS through the use of antioxidants as aforementioned and primarily with ascorbic acid in the dry and or liquid phase is a necessity.

Furthermore, ascorbic acid and its salts supercedes the others not only because of the low oxyreducing tension but also because a) it is included in the USP list #375/14232, R11/98 Ascorbic Acid Injection USP, b) it is used as an injectable medicine in large quantities for the treatment of scurvy and severe burn wounds but can also be used preventively and c) doesn't react or affect the contained ingredients such as LAS and other amino acids such as glycine which is usually contained in injectable LAS products.

The protection of the aforementioned final product from light exposure can be achieved through of the use of (a dark colored) vial with a hollow non-transparent cap (as described in U.S. Pat. No 6,627,192 and in the European Patent EP 1075434). within its hollow cap while below it in the solution of the vial is the injectable liquid. Thus, by removing the outer protective cover of the cap and depressing the exposed piston, the seal is broken releasing the dry phase into the liquid phase where the dilution takes place producing the final injectable product. With the help of i.e. a syringe, the piston can be pierced, to extract the injectable solution which will then be injected to the patient.

Advantages of the Invention that I described and of any other equivalent that I herewith validate are:

1. Protection from oxidations (as described in the above example for injectable LAS)

2. Protection from light exposure by placing the ingredients in a hollow cap and moreover by placing the product within a dark colored vial.

Simultaneous protection from oxidations and exposure to light when within the dark colored vial with the hollow cap an antioxidant factor is added to either the dry phase, liquid phase, or both.

3. The use of injectable, drinkable or other forms with use of one vial instead of two. Currently the dry phase and the liquid phase are separate/stored in two vials.

4. Avoidance of accidental contamination or the complicated and risky process which the use of two vials creates.

5. No need for instant sterilized solutions or water (as in the case of drinkable envelopes)

6. Less use of antioxidants due to the limited space within the hollow cap (air-oxygen).

7. Reduced costs (production, transportation, sterilization, packaging, longer life of the products, safer distant deliveries i.e. to developing countries.

8. Greater safety-profit margin in the entire chain from production to end consumer.

9. Shortened production times, less inventory of required materials, storage spaces, etc.

The above are some of the advantages which this patent offers when it is applied to humans or animals and especially in remote places such as sea voyages, excursions trips, explorations, etc.

Aside from preventive and therapeutic treatments for humans and animals, i.e. antibiotics, vaccines, the proposed system of one container can also be applied to drinkable or injectable antidotes and ointments in the case of bee and other insect stings, snake bites, allergic reactions from marine organisms, etc.

It is apparent that the advantages of the use of antioxidant, light protection and application methods described here separately or in combination, can be applied in a multitude of other cases of substances, medicines, and products which need protection from oxidations, light exposure, or both with the proposed, simpler, safer and more economical production process and application of these final products.

Thus the invention may be very useful, because it simplifies and makes the process safer especially where the existence of a sterilized solution is difficult or impossible. As the present invention suggests, the active ingredient can be ready in the cap of the vial and with one depression, the entire final product that contains sensitive ingredients becomes practical, sterile, and ready for immediate use.

1. Pharmaceutical and/or medicinal product destined for humans or animals, which is applied in an injectable, drinkable, dry or liquid form, which contains at least one active ingredient, and one antioxidant factor that serves to protect from oxidations. Additionally for the protection from exposure to light and UV radiation, these can be placed in special containers (hollow cap, dark colored vial). Needless to say, both protections can be provided to a single product.

2. Product according to claim 1, that has a dry and liquid phase, that has an antioxidant factor in the dry phase or liquid phase or both of the phases.

3. Use of an antioxidant factor in a pharmaceutical and/or medicinal product destined for humans or animals, and the protection of each active ingredient, its excipients and/or its packaging from oxidations, where the choice of the antioxidant factor depends on the type, quantity, and packaging of the active ingredient or its excipients, that require the antioxidant protection we desire.

4. Use according to claim 3, of the addition of an antioxidant factor within the final product or within the package of a container with a special receiver.

5. A receiver of the antioxidant factor according to claim 4, which is permeable in order to bond the oxygen/oxy free radicals contained within the package of the product necessitating protection.

6. The container of the antioxidant factor according to claim 4 can be in the form of either an envelope or capsule that is air permeable in order to bond the oxygen/oxy free radicals contained within the package of the product necessitating protection.

7. Antioxidant factor according to claim 1 which is chosen from: ascorbic acid and its salts (vitamin C), glutathione (GSH), Vitamin E, Uric acid, Urea, L-phenylalanine, L-histidine, Salicylic acid, Acetylsalicylic acid (aspirin), DMSO (dimethylsulfoxide), Mannitol, Tocopherols, Reducing enzymes such as SOD (superoxidase dismutase), Glutatnione reductase, Catalase, Amino acids or reducing micropeptides, Cysteine, Cystine, Glutamine, Glutaminic, Cytochromes, Selinium (Se) and its bonds, Chelate bonds such as EDTA which bond iron ions Fe++ (Fenton reaction) and copper Cu++, or a combination of the above.

8. Container for the product according to claim 1, which has a non transparent hollow cap for the preservation of the active ingredient, its excipients, and of the antioxidant factor for the protection of the final product from oxidations due to light exposure and UV radiation, as well as a dark colored vial for the protection of the solution

9. Use of a vial according to claim 8, for the injectable application of the final product which is sensitive to alterations such as LAS lysine acetylsalicylate, in which as described only one vial is necessary.

10. Use of a container according to claim 8, for the application of medicines such as antibiotics, vaccines, antidotes that up until now required two separate vials for keeping the dry and liquid phase separate, in which only one container is now necessary, making it ideal for simpler, easier, and safer use in remote areas, sea excursions, and military expeditions.
